(19) United States
(12) Patent Application Publication
(10) Pub. No.: US 2009/0081138 A1
(43) Pub. Date: Mar. 26, 2009

(54) CANCER CHEMOPROTECTIVE COMPOSITIONS AND NATURAL OILS AND METHODS FOR MAKING SAME

(75) Inventor: Kean Ashurst, Taylorsville, KY (US)

Correspondence Address: FOLEY AND LARDNER LLP;SUITE 500, 3000 K STREET NW, WASHINGTON, DC, 20007, US

(73) Assignee: Caudill Seed Company, Incorporated

(21) Appl. No.: 11/908,728

(22) PCT Filed: Mar. 16, 2006
(86) PCT No.: PCT/US2006/010032

§ 371 Date: Jun. 3, 2008

Related U.S. Application Data
(60) Provisional application No. 60/662,952, filed on Mar. 18, 2005.

(51) Int. Cl. A61K 8/46; A23L 1/212; A61K 8/36; C11B 1/10; A61Q 17/04; A61Q 5/02; A61Q 5/12

(52) U.S. Cl. 424/59; 426/655; 510/119; 554/8

(57) ABSTRACT

Cancer chemoprotective compositions containing reduced oil-content extraction meals made from plants containing natural oils and glucosinolates. The oil content of the extraction meals may be reduced using batchwise or continuous supercritical fluid extractions. Also provided are glucosinolate-rich compositions containing purified glucosinolates isolated from plant materials. The glucosinolate-rich compositions may be made by reducing the oil content of a plant materials containing natural oils and glucosinolates and isolating the glucosinolates from the reduced oil-content plant materials using a membrane extraction. Natural oils containing isothiocyanates are also provided. The natural oils are well-suited for use in skin and hair care products.

### FIELD OF THE INVENTION

This invention relates to cancer chemoprotective compositions having a high glucosinolate content and low oil content and to methods for producing the compositions from plant materials using supercritical extraction. High purity, natural oils containing isothiocyanates obtained through the supercritical extractions are also provided.

### BACKGROUND

Numerous studies have shown that eating certain vegetables, particularly cruciferous vegetables, may reduce one's risk of developing cancer. The origin of this chemoprotective effect is generally attributed to glucosinolates in the vegetables that are converted into isothiocyanates by contact with endogenous myrosinase enzymes when plant cell walls are breached. Some of these isothiocyanates have been shown to be potent Phase II enzyme inducers, which can protect cells against the toxic and neoplastic effects of carcinogens.

In attempts to isolate glucosinolates and isothiocyanates from plants, various organic solvent and aqueous extraction methods have been developed. These methods generally entail extracting plant material in solvents, such as water, dimethyl sulfoxide, dimethylformamide, acetonitrile, or a combination thereof. Unfortunately, these solvent-based extractions have met with limited success in extracting high concentrations of glucosinolates from plant materials. Typically, the concentration of glucosinolates in the glucosinolate-containing extract is less than 2 wt. %. In addition, these extraction processes typically leave significant amounts (e.g., ≦5 wt. %) of oil in the extraction meal. This is particularly problematic for plants whose natural oils contain erucic acid, which has been shown to cause heart lesions. For this reason, extraction meals containing significant amounts of erucic acid are unsuitable for use as food additives. Another problem associated with presently available glucosinolates isolated from cruciferous plants is that they have plant proteins associated therewith. The presence of plant proteins may render the isolated glucosinolates non-hypoallergenic and creates excessive foam and sticky conditions upon rehydration of extracts containing the glucosinolates. Thus, a need exists for an extraction meal having a high glucosinolate content and a low oil content, for use as a chemoprotective food additive. In addition, a need exists for isolated glucosinolates that are free of plant proteins.

### SUMMARY

One aspect of the present invention provides reduced oil-content extraction meals made from plants containing natural oils and glucosinolates and methods for making the same. The reduced oil-content extraction meals provide cancer chemoprotective compositions and may be used as food additives in food products or as dietary supplements. The reduced oil-content extraction meals may be ingestible, all-natural and free of chemical additives. The oil content of the extraction meals may be reduced using batchwise or continuous supercritical fluid extractions.

Another aspect of the invention provides glucosinolate-rich compositions containing purified glucosinolates isolated from plant materials and methods for making the same. Like the reduced oil-content extraction meals, the isolated glucosinolates provide cancer chemoprotective compositions and may be used as food additives in food products or as dietary supplements. The glucosinolate-rich compositions may be ingestible, all-natural and free of chemical additives. The glucosinolate-rich compositions may be made by reducing the oil content of plant materials containing natural oils and glucosinolates and isolating the glucosinolates from the reduced oil-content plant materials. In some embodiments the reduced oil-content extraction meals provided herein may be subjected to a membrane extraction process which isolates glucosinolates by molecular weight and eliminates plant proteins.

Yet another aspect of the invention provides natural oils containing isothiocyanates, such as sulforaphane, extracted from plant materials. These oils may be obtained through a supercritical extraction of a mixture of plant materials with myrosinase enzymes. The natural oils are well suited for use in skin and hair care products, including lotions, sunscreens, shampoos and conditioners.

Preferred plant starting materials for making the cancer chemoprotective compositions the natural oils include plants from the Cruciferae and Brassicaceae families including, but not limited to, broccoli and field pepperweed.

Further objects, features and advantages of the invention will be apparent from the following detailed description when taken in conjunction with the accompanying drawings.

### BRIEF DESCRIPTION OF THE DRAWINGS

FIG. 1 shows a process flow diagram for a method of obtaining a reduced oil-content extraction meal using a batchwise supercritical extraction.

FIG. 2 shows a process flow diagram for a method of obtaining a reduced oil-content extraction meal using a continuous supercritical extraction.

FIG. 3 shows a process flow diagram for a method of obtaining purified glucosinolates from a reduced oil-content extraction meal.

FIG. 4 shows a schematic diagram of a system that may be used to produce a reduced oil-content extraction meal and a high purity natural oil containing isothiocyanates in accordance with the present invention.

### DETAILED DESCRIPTION

A number of terms are used repeatedly in the following description of the present invention. The following definitions are provided to assist in the understanding of the invention.

A bifunctional inducer is a molecule which increases activities in both Phase one enzymes and Phase two enzymes and requires the participation of aryl hydrocarbon (Ah) receptor and its cognate Xenobiotic Response Element (XRE).

A cancer chemoprotective compound is a chemical agent that reduces susceptibility in a mammal to the toxic and neoplastic effects of carcinogens.

A dietary supplement is a composition consumed in order to supplement the diet for a nutritional purpose. Dietary supplements may take a variety of forms including but not limited to pills, capsules, tablets and liquid forms.

A food additive is a composition intended to be added to a food product in order to enhance the nutritional value of a diet that includes said food product.

An extraction meal is any material remaining after one or more compounds has been extracted from a starting material via an extraction with solvent. For example, the plant materials remaining after natural oils have been extracted in a supercritical fluid extraction compose an extraction meal.

A food product is any ingestible preparation containing the compositions of the present invention. The food product may be fresh or processed and may be a solid or liquid food. For the purposes of this disclosure, pharmaceutical compositions such as pills and tablets are included within the definition of food products. As used herein, the term “ingestible” means that up to specified amounts of the preparation can be ingested by a human without generally causing negative health effects. Examples of ingestible preparations include those compounds “generally recognized as safe” (“GRAS”) by the United States Food and Drug Administration (“FDA”). In particular, ingestible compounds include those compounds listed as approved under 21 C.F.R. §§ 73, 74, 172, 182 and 184.

A monofunctional inducer increases the activity of Phase two enzymes selectively without significantly affecting the activity of Phase one enzymes.

Plant materials are whole plants or parts of plants that include at least some plant solids. Plant stems, seeds (including hulls and/or meat), seedlings, sprouts, florets, leaves, roots, rosettes and flowers are examples of plant parts from which plant materials may be composed.

Ingestible cancer chemoprotective compositions including low oil content and high glucosinolate content plant materials and purified or partially purified glucosinolates extracted and isolated from plant materials are provided. The cancer chemoprotective compositions are desirably all natural and may be used as food additives and dietary supplements or may be included in various foods to produce cancer chemoprotective food products. The food additives, dietary supplements and food products may be used to increase the chemoprotective amount of Phase 2 enzymes in a mammal or to reduce the level of carcinogens in a mammal by administering an effective quantity of the food additives, dietary supplements or food products to the mammal. Methods for producing the cancer chemoprotective compositions are also provided.

The glucosinolates present in the compositions (e.g., glucoraphanin) are metabolic precursors of isothiocyanates, such as sulforaphane. Conversion of the glucosinolates to isothiocyanates occurs via hydrolysis of the glucosinolates by myrosinase which may be present in the plant materials or added to the compositions as exogenous myrosinase. The isothiocyanates are inducers of Phase 2 enzymes which are known to detoxify carcinogens in mammalian cells. The glucosinolates are preferably those that convert into monofunctional inducers, such as alkylthioglucosinolates. Such glucosinolates include glucoraphanin, glucoerucin, glucoiberin. However, glucosinolates that are bifunctional inducers, such as indole glucosinolates, may also be present. Such glucosinolates are desirably limited to amounts that would not negatively impact the nutritious qualities of food additives, dietary supplements and/or food products containing them.

The cancer chemoprotective compositions and extracted natural oils may be made from plants containing natural oils and glucosinolates. Although any plants containing both natural oils and glucosinolates may be used as a starting material, plants belonging to the Cruciferae family are well-suited for use in making the present compositions because they are known to contain substantial quantities of glucosinolates. Within the Cruciferae family, those plants belonging to the genus Brassica are particularly well-suited for use as starting materials. Preferred members of the Brassica genus are brassica oleracea selected from the group of varieties consisting of acephala (kale, collards, wild cabbage, curly kale), medullosa (marrowstem kale), ramosa (thousand head kale), alboglabra (Chinese kale), botrytis (cauliflower, sprouting broccoli), costata (Portuguese kale), gemmifera (Brussels sprouts), gongylodes (kohlrabi), italica (broccoli), palmifolia (Jersey kale), sabauda (savoy cabbage), sabellica (collards), and selensia (borecole), among others. Particularly useful broccoli cultivars to be used in the claimed method are Saga, DeCicco, Everest, Emerald City, Packman, Corvet, Dandy Early, Emperor, Mariner, Green Comet, Green Valiant, Arcadia, Calabrese Caravel, Chancellor, Citation, Cruiser, Early Purple Sprouting Red Arrow, Eureka, Excelsior, Galleon, Ginga, Goliath, Green Duke, Greenbelt, Italian Sprouting, Late Purple Sprouting, Late Winter Sprouting White Star, Legend, Leprechaun, Marathon, Mariner, Minaret (Romanesco), Paragon, Patriot, Premium Crop, Rapine (Spring Raab), Rosalind, Salade (Fall Raab), Samurai, Shogun, Sprinter, Sultan, Taiko, and Trixie. However, many other broccoli cultivars are suitable. Particularly useful cauliflower cultivars are Alverda, Amazing, Andes, Burgundy Queen, Candid Charm, Cashmere, Christmas White, Dominant, Elby, Extra Early Snowball, Fremont, Incline, Milkyway Minuteman, Rushmore, S-207, Serrano, Sierra Nevada, Siria, Snow Crown, Snow Flake, Snow Grace, Snowbred, Solide, Taipan, Violet Queen, White Baron, White Bishop, White Contessa, White Corona, White Dove, White Flash, White Fox, White Knight, White Light, White Queen, White Rock, White Sails, White Summer, White Top, Yukon. However, many other cauliflower cultivars are suitable.

The cancer chemoprotective compositions and extracted natural oils may also be made from plants of the Brassicaceae family. Within the Brassicaceae family, certain plants belonging to the genus Lepidium L. are well-suited for use as starting materials. Lepidium campestre L. (field pepperweed) is one such plant. Recent studies have shown that field pepperweed harvested at the early stage are particularly rich sources of glucosinolates.

The plant materials used to make the cancer chemoprotective compositions and the extracted natural oils may take the form of whole plants or plant parts including, but not limited to, stems, roots and florets. The plant materials may come from plants in any stage of development, including mature plants, sprouts, seedling, seeds or a mixture thereof. However, the use of sprouts or seeds as starting materials is desirable because sprouts and seeds are a particularly rich sources of glucosinolates and, in particular, glucosinolates that are precursors to isothiocyanates that induce the activity of Phase 2 enzymes, without inducing biologically significant activities of those Phase 1 enzymes that activate carcinogens.

One aspect of the invention provides low oil content plant materials from which natural oils have been extracted. The extracted oils are often a valuable product in their own right and, therefore, the efficient extraction of oils from the plant materials is desirable from a profitability standpoint. However, the extraction of natural oils from the plant materials may also be desirable because some oils, such as canola and rapeseed oil, naturally found in glucosinolate-containing plants contain erucic acid, which has been found to cause heart lesions. In some embodiments of the invention, the plant materials used to produce the compositions and natural oils are not canola plant materials or rapeseed plant materials. In addition, the removal of oils from the plant materials facilitates the extraction and isolation of glucosinolates and other compounds from the plant materials because the oils tend to clog filtering equipment, such as membranes, commonly used in extractions.

In one embodiment, these low oil content plant materials take the form of an extraction meal containing no more than about five wt. % residual oil. This includes embodiments where the extraction meal contains no more than about 4 wt. %, no more than about 3 wt. %, no more than about 2 wt. % and no more than about 1 wt. % residual oil. In other embodiments, the extraction meal is substantially free of or free of oils, where the meal is considered to be substantially free of oil if it contains, for example, no more than about 0.1 wt. % oil.

While natural oils are extracted from the plant materials, the glucosinolates present in the plant materials are retained or substantially retained. As a result, the extraction meal is a glucosinolate-rich plant material. In some embodiments, the extraction meal contains at least about 2 wt. % glucosinolates. This includes embodiments where the extraction meal contains at least 3 wt. %, at least 4 wt. %, at least 5 wt. % or at least 5.5 wt. % glucosinolates. In some embodiments of the invention the extraction meal is free of or substantially free of glucosinolates that are precursors to compounds that are Phase 1 enzyme inducers.

The low oil content plant materials may be produced using a supercritical fluid extraction to remove natural oils from the plant starting materials. The inventor has discovered that the use of a supercritical fluid extraction makes it possible to extract significantly more oil from the plant materials than the pressing techniques which have traditionally been used. The supercritical extraction may be conducted in a batch-wise or continuous manner. The supercritical extraction may be performed by passing a supercritical fluid (SCF) through the plant starting material under temperature and pressure conditions that render natural oils in the plant materials soluble in the supercritical fluid. As the fluid passes through the plant starting materials, it diffuses into the pores of the plant material matrix and solubilizes natural oils. The oils are then carried away from the plant materials by the supercritical fluid. The supercritical fluid extract containing the oils is then collected while the plant materials are left behind as the extraction meal. The oils in the oil-containing extract may be collected by adjusting the temperature and/or pressure of the supercritical fluid to render the oils insoluble. The extraction may be continued until a sufficient amount of oil has been removed from the plant materials being processed.

In an alternative aspect of the invention, the supercritical extraction may be optimized to produce a natural oil containing isothiocyanates, rather than a glucosinolate-rich extraction meal. This may be accomplished by including myrosinase with the plant material in the extraction. This is accomplished by adding exogenous myrosinase, to the plant material. Myrosinase may be obtained from diakon radish. (Although the plants naturally contain myrosinase, the natural myrosinase is generally deactivated prior to the extraction process in order to prevent uncontrolled conversion of the glucosinolates into isothiocyanates, which are unstable in the presence of water.) The addition of myrosinase to the plant material results in the conversion of the glucosinolates, which are insoluble in oil, into isothiocyanates, which are soluble and stable in oil. Thus, in the supercritical extraction, the oil fraction will contain isothiocyanates. In some embodiments, the present methods produce natural oils, such as broccoli oils, having an isothiocyanate (e.g., sulforaphane) content of at least 0.3%. This includes embodiments where the oils have an isothiocyanate content of at least 0.4% and further includes embodiments where the oils have an isothiocyanate content of at least about 0.5%.

The natural oils removed from the plant materials may be used, for example, in skin and hair care products. Broccoli oil, including broccoli oil derived from broccoli spouts and/or mature plants, is particularly well-suited for use in skin and hair care products. Such products include, but are not limited to, lotions, sunscreens, shampoos and conditioners. Other ingredients that may be included in the skin and hair care products include, but are not limited to, other natural and essential oils, emulsifiers, emollients, antioxidants, UV and/or IR protection factors and fragrances. Suitable ingredients of this type are described in U.S. Patent Application Publication No. 2003/0091518, the entire disclosure of which is incorporated herein by reference.

Some advantages realized by using a supercritical fluid extraction, as compared to a more conventional liquid solvent extraction, are as follows: 1) the higher diffusion coefficients, lower viscosities and absence of surface tensions in supercritical fluids relative to liquid allows for better penetration into the pores of the plant materials and higher extraction efficiencies; 2) extraction selectivity may be readily tailored by varying the temperature and pressure in a supercritical extraction to alter the solubility of the various components in the supercritical fluid; and 3) supercritical extractions do not leave a chemical residue.

The supercritical fluids used in the extraction are desirably nontoxic and nonexplosive. Examples of fluids that may be used in the extractions include, but are not limited to, carbon dioxide (CO₂), ethane, ethylene and water. Carbon dioxide is a particularly desirable supercritical fluid due to its low critical parameters (31.1° C., 1070 psi), which helps prevent thermal degradation of plant starting materials when they are being extracted. Also advantageous are its low cost and nontoxicity, nonflammability and noncorrosiveness. The use of CO₂ as an extraction fluid allows for the production of cancer chemoprotective compositions that are all-natural, organic and generally recognized as safe (“GRAS”) by the United States Food and Drug Administration (“FDA”).

Supercritical fluid extractions offer several advantages over more conventional extractions that have been used to isolate glucosinolates from plant materials. For example, the dissolving power of a supercritical fluid may be controlled by changing the pressure and/or temperature of the fluid in order to optimize the extraction of the desired oils. In addition, the supercritical fluid is easily recoverable from the extract due to its volatility. Finally, nontoxic solvents (e.g., CO₂) are available for supercritical fluid extractions to produce products without harmful residues.

Supercritical fluid extraction systems suitable for producing the cancer chemoprotective compositions and extracted natural oils using a batch-wise extraction are well known and commercially available. One such system is described in U.S. Pat. No. 6,737,552, the entire disclosure of which is incorporated herein by reference.

Typical pressure and temperature ranges for a batch-wise supercritical fluid extraction using carbon dioxide as the supercritical fluid are about 4000 to 4500 psi (e.g., about 4250 psi) and about 120 to 180° F. (e.g., about 150° F.). The duration of the supercritical fluid extraction will vary depending on the acceptable amount of residual oil in the extraction meal, however batch-wise supercritical extraction times of about 12 to 24 hours are generally sufficient to provide a low oil content extraction meal.

The supercritical fluid extractions may also be conducted in a continuous manner. In the continuous extractions, the starting plant materials are pressed in the presence of an extraction agent under sufficient pressure to produce a supercritical fluid. For example, the starting plant materials may be pressed in a screw press with a simultaneous injection of an extraction agent, such as carbon dioxide, under enough pressure to provide a supercritical fluid extraction. An apparatus suitable for conducting a continuous supercritical fluid extraction of glucosinolates in accordance with the present invention is described in U.S. Pat. No. 5,939,571, the entire disclosure of which is incorporated herein by reference. Briefly, the apparatus includes an inlet for introducing the starting plant materials into a substantially cylindrical pressing body having an outlet for discharging the extraction meal. The pressing body is sealed by a jacket and contains a press screw for pressing the plant materials as they pass through the pressing body. The press screw and/or pressing body include outlets through which an extraction agent may be introduced under pressure into the pressing body where it penetrates the plant materials. The oil-containing extraction fluid along with oil that has been pressed from the plant materials is then passed out of the pressing body through oil outlets. Using this apparatus, a continuous flow of starting plant materials may be exposed to a continuous flow of the supercritical extraction fluid to provide an oil-containing extract and a low oil content extraction meal.

Typical pressure and temperature ranges for a continuous supercritical fluid extraction using carbon dioxide as the supercritical fluid are: about 3000 to 6000 psi and about 180 to 230° F. (e.g., about 195 to 210° F.). The duration of the supercritical fluid extraction will vary depending on the acceptable amount of residual oil in the extraction meal, however continuous supercritical extraction times of about 1 to 5 minutes (e.g., about 2 minutes) are generally sufficient to provide a low oil content extraction meal.

In some embodiments, the supercritical fluid extractions may be conducted on raw plant materials that have undergone little or no pre-extraction processing. The desirability of pre-extraction processing depends, at least in part, on whether myrosinase enzymes present in the starting plant materials will be deactivated during the extraction and on whether the desired product is a glucosinolate-rich extraction meal or an isothiocyanate-containing natural oil. If active endogenous myrosinase enzymes are present when cells walls in the plant materials are breached in the enzymes will convert the glucosinolates into their corresponding isothiocyanates. Unfortunately, many beneficial isothiocyanates are unstable once formed in the presence of water. Therefore, if the desired product is a glucosinolate-rich extraction meal, it is advantageous to deactivate the endogenous myrosinase enzymes in the starting plant materials before they begin to convert the glucosinolates. If a batch-wise supercritical fluid extraction is to be used, myrosinase enzymes present in the plant materials will typically be deactivated during the extraction process obviating a pre-extraction processing step for deactivating the myrosinase enzymes. Thus, in a batch-wise supercritical extraction the raw plant starting materials may be introduced into the supercritical fluid extraction system either in whole form or after a grinding, milling, chopping and/or flaking step.

Where a continuous supercritical fluid extraction is to be used, however, it may be advantageous to cook or otherwise thermally treat the plant materials in order to deactivate the myrosinase enzymes prior to the extraction process. For example, in one typical pre-extraction processing step the starting plant materials may be crushed (e.g., in a roller mill) and heat treated in a cooker under regulated moisture conditions at a temperature sufficient to deactivate the myrosinase enzymes. The cooked plant materials may then be extruded into a plant meal prior to being introduced into the continuous supercritical fluid extraction apparatus.

Although it is not necessary, the starting plant materials may be pressed (e.g., in an expeller press) in order to press out some of the natural oils and this press cake may be used in a subsequent supercritical fluid extraction in order to further reduce the oil content of the plant materials.

Optionally, the moisture content of the starting materials may be reduced (e.g., by drying or dehydrating) prior to the oil extraction process. The moisture content of the starting plant materials may be higher if an isothiocyanate-containing natural oil, rather than a glucosinolate-rich extraction meal, is the desired end product. For example, the plant materials are generally dried to a water content of no more than about 6 wt. % prior for the production of a glucosinate-rich extraction meal. However, the plant materials having a water content of up to 20 wt. %, or even higher, may be used in the production of an isothiocyanate-containing natural oil.

The low oil content extraction meal may be used “as is” as a food additive or as a dietary supplement. Alternatively, the extraction meal may undergo various post-extraction processing steps. For example, these processing steps may include one or more of the following: removal of some plant solids (e.g., seed coats) from the extraction meal, drying, and grinding. The processed or unprocessed extraction meal may be combined with a suitable carrier and may be provided in powder or tableted form. Suitable carriers with which the extraction meal may be combined include but are not limited to starches and sugars.

In addition to glucosinolate-rich plant materials, the present invention provides materials containing purified natural glucosinolates isolated from plant materials. In some embodiments, these materials are free of or substantially free of plant proteins. For example, these glucosinolate-rich materials may contain isolated glucosinolates and sugars retained from the plant materials, without the accompanying proteins. The elimination of proteins from the isolated glucosinolates is advantageous for at least two reasons. First, the elimination of proteins provides a hypoallergenic product. Second, the elimination of proteins eliminates foaming upon rehydration. Conventional products containing isolated natural glucosinolates retain the proteins and include chemical additives to eliminate the foam. The present methods eliminate the need for such chemical additives.

Like the glucosinolate-rich plant materials provided herein, the materials containing the isolated glucosinolates may be all-natural, organic, GRAS and/or free of chemical additives. In some instances the glucosinolate-rich materials contain at least about 5 wt. % glucosinolates. This includes glucosinolate-rich materials containing at least about 7 wt. % glucosinolates, further includes glucosinolate-rich materials containing at least about 10 wt. % glucosinolates, still further includes glucosinolate-rich materials containing at least about 15 wt. % glucosinolates and even further includes glucosinolate-rich materials containing at least about 20 wt. % glucosinolates. The glucosinolates are desirably glucoraphanins, however, is some preferred embodiments the glucosinolates may also include other beneficial glucosinolates in lesser quantities.

Generally, the isolation of the glucosinolates from plant materials is carried out by reducing the natural oil content of the plant materials to provide a reduced oil-content plant meal and then isolating glucosinolates from the plant meal using a separation step. A preferred separation step is a membrane extraction designed to isolate glucosinolates and, optionally, sugars from the plant materials while eliminating plant proteins.

For the purposes of this disclosure, a reduced oil-content plant meal is any plant material from which natural oils have been partially or completely removed. In some preferred embodiments, the reduced oil-content plant meal contains no more than about 10 wt. % natural oils. This includes embodiments where the plant meal contains no more than about 5 wt. % natural oils and further includes embodiment where the plant meal contains no more than about 3 wt. % natural oils. For example, the low oil-content extrusion meals described herein may be used as the low oil-content plant meals from which glucosinolates are isolated. The use of a reduced oil content plant meal represents a significant advantage, because the plant materials naturally contain enough oil to clog many separation devices, including membranes, rendering the isolation of glucosinolates from the plant materials impossible.

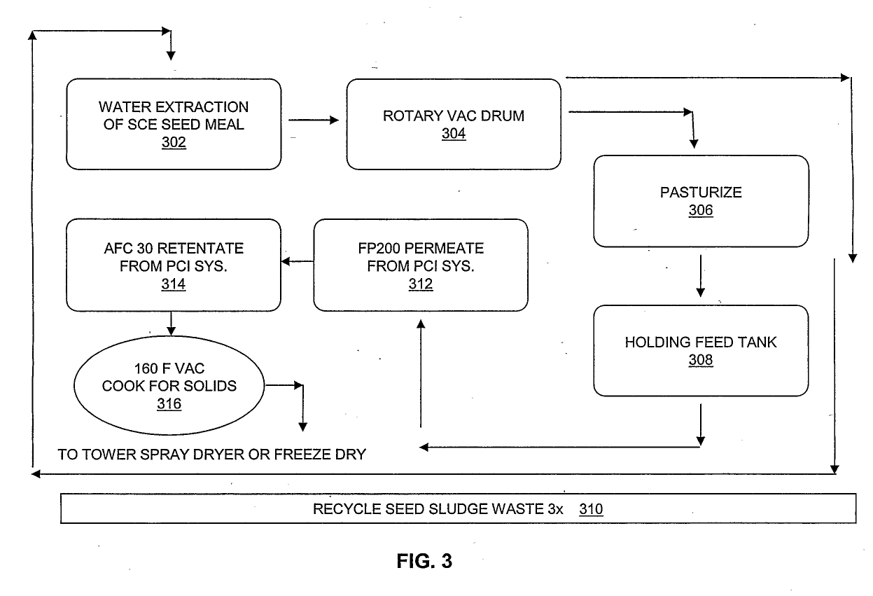

In a typical isolation procedure (shown in FIG. 3) the low oil-content plant meal is first subjected to an extraction in an aqueous or organic solvent to provide a glucosinolate-containing extraction fraction 302. The temperature of the solvent in this initial extraction may be selected to maximize the solubility of the glucosinolates of interest. Glucosinolates in this extraction fraction are then isolated. Optionally, any residual plant solids remaining in the glucosinolate-containing extraction fraction may be removed by filtering prior to the isolation step. For example, the extraction fraction may be passed through a rotary vacuum drum 304. In addition, the extraction fraction may be pasteurized 306 and passed into a holding tank 308 before undergoing a membrane separation. The filtered plant solids, along with the plant solids (or sludge) left over from the aqueous or organic extraction may be recycled in a subsequent extraction 310. This recycling of the plant materials may be conducted multiple times to maximize the glucosinolate yield.

In a preferred embodiment, a membrane separation may be used to isolate the glucosinolates from the extraction fraction. Suitable membrane separation techniques include reverse osmosis and ultrafiltration. In one embodiment the membrane separation is a two step separation in which the first membrane is a protein filtering membrane having a porosity sufficient to allow the passage of glucosinolates and sugars but not proteins 312. For example, an ultrafiltration membrane could be employed in the first separation. Such membranes desirably have a molecular weight cutoff of about 100,000 Daltons. This first membrane separation optionally may include a diafiltration step in order to maximize the glucosinolate yield. The permeate from this first separation then undergoes a second membrane separation wherein a glucosinolate-retaining membrane has a porosity sufficient to allow the passage of water and salts but not glucosinolates 314. For example, a nanofiltration membrane could be employed in the second separation. The resulting glucosinolate-rich retentate provides a glucosinolate-rich material comprising glucosinolates (e.g., glucoraphanin) and, optionally, natural sugars.

Membranes for use in the isolation of the glucosinolates are commercially available. For example, suitable ultrafiltration membranes include, but are not limited to, ultrafiltration and nanofiltration membranes available from PCI Membrane Systems Inc. (Milford, Ohio). Specific examples of suitable protein-filtering and glucosinolate-retaining membranes are PCI's FP 200 membrane (a polyvinylidene fluoride ultrafiltration membrane) and AFC 30 membrane (a polyamide thin film composite nanofiltration membrane), respectively.

The glucosinolate-rich retentate from the membrane extraction optionally may be subjected to a final vacuum heating step and/or a drying step, such as a spray drying or freeze drying, in order to provide a dried product 316. This product typically will take the form of a powder which rehydrates in water without visible residue. Like the glucosinolate-rich extraction meals provided herein, the glucosinolate-rich materials containing isolated glucosinolates may be used “as is” as a food additive or as a dietary supplement or may be added to a food product in order to provide a cancer chemoprotective food product.

### EXAMPLES

### Example 1

### Production of a Glucosinolate-Rich Extraction Meal Using a Batchwise Supercritical Extraction.

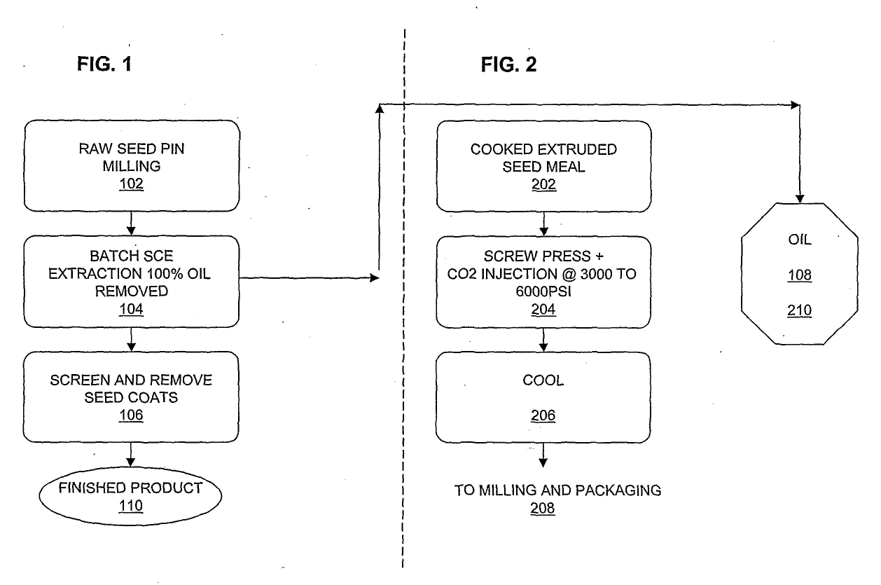

A sample process flow diagram for a method of obtaining a reduced oil-content extraction meal using a batchwise supercritical extraction is shown in FIG. 1. A glucosinolate-rich extraction meal may be made from flaked rolled seed (e.g., broccoli seed) material made by flaking raw seeds in a flaking mill equipped with a pin feeder 102. This material is translucent in appearance and typically has an oil content of about 30 wt. % to 35 wt. %.

The flake/rolled seed is heated to 190° F. for a maximum of 3 min. utilizing a thermal screw conveyor in order to deactivate myrosinase enzymes and kill microorganisms. This “kill step” may be even shorter. For example, in some instances the plant materials may be heated to a temperature of at least about 180° F. for 1 minute of less. The material is then introduced into the batch extraction chamber and undergoes supercritical extraction with CO₂ at a temperature of 150° F. and 4000 psi 104. Pumps circulate the CO₂ through the material and the material is captured on screens and the seed coats are removed 106. Temperature pressure and CO₂ flow rate are controlled and monitored with a programmable logic controller. The heat in the thermal screw can be dry or wet depending on the desired product. Glucoraphanins are produced using a dry heat and sulforaphane is produce using a wet heat with the addition of enzymes. The oil from the supercritical extraction is removed from the reaction vessel and collected 108. The sulforaphane can be infused into the oil fraction using a separate process.

The reduced oil-content extraction meal on discharge is a white powder having an oil content of no more than ½% and a moisture content of no more than 2% 110.

### Example 2

### Production of a Glucosinolate-Rich Extraction Meal Using a Continuous Supercritical Extraction.

A sample process flow diagram for a method of obtaining a reduced oil-content extraction meal using a batchwise supercritical extraction is shown in FIG. 2. A glucosinolate-rich extraction meal may be made from cooked, extruded plant material 202. The cooked, extruded plant material is fed into and pressed in a screw press with a simultaneous injection of carbon dioxide at a pressure of about 3000 to 6000 PSI 204. The extraction meal exiting the screw press is allowed to cool 206 and may be milled into a powder and packaged 208. The oil from the supercritical extraction is removed from the reaction vessel and collected 210.

### Example 3

### Production of An Isothiocyanate-Containing Natural Oil Using a Supercritical Extraction and a Double Oil Separation.

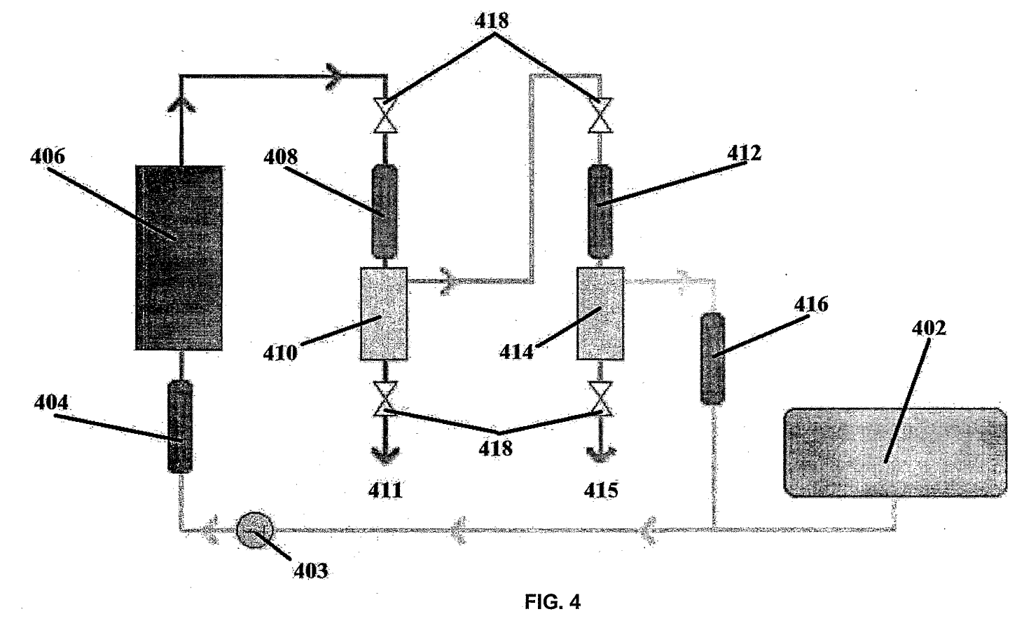

A sample apparatus and method for producing a glucosinolate-rich extraction meal using a supercritical extraction and a double oil separation is shown in FIG. 4. In this method liquid CO₂ from a storage vessel 402 is pumped, by a pump 403, through a heater 404 under pressure to fonn supercritical C0₂ which is fed into an extraction vessel 406 containing a plant material comprising glucosinolates and oil. For example, extraction vessel 406 may contain dried (e.g., to a water content of about 20 wt. %), heat-treated, one-day-old broccoli sprouts that have been processed by wet-milling. Myrosinase may is also added to extraction vessel 406 in order to convert glucosinolates in the plant material into isothiocyanates which dissolve in and are stabilized by the natural plant oils. The first fraction with the supercritical CO₂ carrying the natural oils from the plant material passes out of extraction vessel 406 and through an evaporator 408 and a separator 410 to isolate and collect the natural oils 411. A second fraction of CO₂ is then passed through a second evaporator 412 and separator 414 to isolate and collect more natural oils 415. The CO₂, now in gaseous form, is then passed into a condenser 416 and may be re-routed through the system again. Valves 418 may be located along the flow path of the extraction to direct the fractions, extractants and extracts, as needed.

### Example 4

### Production of a Glucosinolate-Rich Extraction Meal Using a Supercritical Extraction with Pre-Processing of Plant Materials.

This example describes one method for pre-processing broccoli seed and sprouts for a supercritical extraction.

Raw Material: The PVP variety Broccoli seeds (Brassica oleracea italica) were cleaned and inspected in California utilizing GAP (Good Agriculture Practices). The seeds were packaged in 50 pound woven nylon bags and palletized from shipment and distribution from CSC-Louisville, Ky.

Sprouting: Upon receipt, the seeds were prepared for sprouting by placing 24 pounds of seed in a 200 mesh nylon zippered bag. The bags were placed on a stainless seal wire frame attached to chemical seed washer. The seeds were submerged in the sanitizing solution and washed and sanitized using a 1% solution of Tsunami-100 for 25 min. (if this material is not approved in a particular country a similar material may be employed.) The bags of sanitized seeds were removed from the seed washer and submerged for soaking at 80°0 F. for 8 hours. Upon completion of the soaking process the bagged seeds were placed on trays for sprouting. Water temperatures should be maintained at 72° F. for 24 to 30 hours or until 80% of the seeds have initiated tail growth. The bags of 1-Day Sprouts were immersed in chilled sanitized water at (36° F. to 38° F.) to retard the growth rate. When the sprout temperature has reached a core temperature of 40° F. the sprouts were transported in a pre chilled refrigerated truck for immediate heat treatment and drying. All transportation and handling of the in-process material were carried out at 40° F. or less.

Enzyme Deactivation and Heat Treatment: The seeds contained myrosinase enzymes that degrade glucoraphanin in an un-controlled manner to sulforaphane. In addition the sprouting process encourages the growth of micro organisms and they are desirably minimized to an acceptable level during the process. The heat treatment was applied as follows. The 1-Day Sprouts were received in bags and emptied into a Chester Jensen (or equivalent) steam jacket cooking kettle with dual counter rotation agitation. Steam was applied to the product until the recorder controller temperature reached 190° F. and this temperature was maintained for a period of three (3) minutes. In some instances extending the heating time to increase the solubility of the final product may be desirable. The product was discharged through a 4 inch outlet in the bottom of the cooking kettle to a Waukesha PD Pump and discharged into hopper of a wet mill.

Wet Milling: Wet milling was accomplished by means of a C.S. Bell La Milpa Grist Mill. The mill wheels were adjusted to the correct thickness and the product was discharged onto the dryer belt.

Drying: Initial temperature of the product entering the dryer was about 140° F. The dryer configuration was such that the internal temperature of the Broccoli plant material was dried to a finished product moisture of not greater than about 6% and cooled to 110° F. at the time of packaging in fiber drums lined with 6 mil poly bags.

Supercritical Extraction: Supercritical extraction in CO₂ was carried out at 400 bar (5800 psi) at 60° F. This process removed 99.5% of the oil from the extraction meal. The resulting percent oil was less than 0.75% of the total weight of the extraction meal. After the SCE process the product was roller milled to the desired particle size.

Packaging: The product was either packaged in poly-lined 55 gallon fiber drums, or hermetically sealed containers under vacuum or gas flushed with CO₂.

For the purposes of this disclosure and unless otherwise specified, “a”, or “an” means “one or more”. All patents, applications, references and publications cited herein are incorporated by reference in their entirety to the same extent as if they were individually incorporated by reference.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member.

While the principles of this invention have been described in connection with specific embodiments, it should be understood clearly that these descriptions are made only by way of example and are not intended to limit the scope of the invention.

What is claimed is:

1. An extraction meal comprising plant solids from a plant from the Cruciferae or Brassicaceae families, the extraction meal comprising at least about 2 wt. % glucosinolates and no more than about 3 wt. % oil.

2. The extraction meal of claim 1, wherein the plant is a member of the genus Brassica or the genus Lepidium L.

3. The extraction meal of claim 1, wherein the plant is broccoli.

4. The extraction meal of claim 1, wherein the plant is pepperweed.

5. The extraction meal of claim 3, comprising no more than about 2 wt. % oil.

6. The extraction meal of claim 3, wherein the extraction meal is substantially free of oil.

7. The extraction meal of claim 3, wherein the extraction meal comprises at least about 4 wt. % glucosinolates.

8. The extraction meal of claim 1, wherein the plant solids comprise seeds or sprouts.

9. A food product comprising the extraction meal of claim 1.

10. A plant-derived composition comprising natural glucosinolates extracted from a plant, the composition being free of or substantially free of proteins.

11. The composition of claim 10, wherein the composition is free of proteins.

12. The composition of claim 10, comprising at least about 7 wt. % glucoraphanin.

13. The composition of claim 10, comprising at least about 10 wt. % glucoraphanin.

14. The composition of claim 10, further comprising natural sugars.

15. The composition of claim 10, wherein the composition is substantially free of non-natural ingredients.

16. A food product comprising the composition of claim 10.

17. An oil composition extracted from broccoli plant solids, the composition comprising broccoli oil and isothiocyanates.

18. The oil composition of claim 17, wherein the isothiocyanates comprise sulforaphane.

19. The oil composition of claim 17, wherein the composition comprises at least 0.3 wt. % isothiocyanates.

20. The oil composition of claim 17, wherein the composition comprises at least 0.5 wt % isothiocyanates.

21. A skin or hair care product comprising the composition of claim 17, wherein the product is selected from the group consisting of skin lotions, sunscreens, shampoos, and hair conditioners.

22. A method for removing oil from a plant material comprising broccoli plant solids or plant solids from a plant in the genus Lepidium L, the method comprising performing a supercritical fluid extraction on the plant material to provide an extract comprising oil and an extraction meal comprising glucosinolates.

23. The method of claim 22, wherein the plant material comprises broccoli plant solids.

24. The method of claim 22, wherein the plant material comprises pepperweed plant solids.

25. The method of claim 22, wherein the plant material comprises plant sprouts.

26. The method of claim 23, wherein the extraction meal comprising glucosinolates comprises no more than about 3 wt. % oil.

27. The method of claim 26, wherein the extraction meal comprises at least 2 wt. % glucosinolate.

28. The method of claim 22, further comprising isolating the glucosinolates from the extraction meal.

29. A method for isolating glucosinolates from plant materials comprising oil and glucosinolates, the method comprising reducing the natural oil content of the plant materials to provide a reduced oil-content plant meal and isolating glucosinolates from the low oil-content plant meal using a membrane extraction to provide a glucosinolate-rich material.

30. The method of claim 29, wherein the glucosinolate-rich material is free of or substantially free of plant proteins.

31. The method of claim 29, wherein the membrane extraction comprises extracting the reduced oil-content plant meal in water to provide an aqueous extract, passing the aqueous extract through at least one protein filtering membrane to provide a permeate that is free of or substantially free of plant proteins and passing the permeate through at least one glucosinolate-retaining membrane to provide a glucosinolate-rich retentate.

32. The method of claim 29, wherein the plant material comprises a plant from the genus Brassica or the genus Lepidium L.

33. The method of claim 29, wherein the plant material comprises broccoli.

34. The method of claim 29, wherein the plant material comprises plant spouts.

35. The method of claim 29, wherein the step of reducing the natural oil content of the plant materials comprises conducting a supercritical fluid extraction on the plant materials.

36. A method for extracting an isothiocyanate-containing natural oil from plant material comprising glucosinolates and oil, the method comprising subjecting a mixture of the plant material and exogenous myrosinase to a supercritical extraction.

37. The method of claim 36, wherein the isothiocyanate content in the extracted natural oils is at least about 0.3 wt. %.

38. The method of claim 36, wherein the natural oils comprise broccoli oil.
